(19) United States
(12) Patent Application Publication
(10) Pub. No.: US 2026/0335511 A1
(43) Pub. Date: Oct. 29, 2026

(54) PROGRAMMED CONTROLLABLE SUSTAINED-RELEASE BIONIC SPINAL CORD GRAFT AS WELL AS PREPARATION METHOD AND APPLICATION THEREOF

(71) Applicant: NANTONG UNIVERSITY, Nantong (CN)

(72) Inventors: Xiaosong GU, Nantong (CN); Yumin YANG, Nantong (CN); Wei SHI, Nantong (CN); Songlin ZHOU, Nantong (CN); Leilei GONG, Nantong (CN); Meiyuan LI, Nantong (CN); Tianmei QIAN, Nantong (CN); Xiu DAI, Nantong (CN); Jiahui GUO, Nantong (CN)

(21) Appl. No.: 19/702,785

(22) Filed: Jun. 9, 2026

Related U.S. Application Data
(63) Continuation of application No. PCT/CN2025/097312, filed on May 27, 2025.

(30) Foreign Application Priority Data
Apr. 25, 2025 (CN) 202510527709.5

(51) Int. Cl. A61L 27/54; A61L 27/22; A61L 27/24; A61L 27/36; A61L 27/38

(52) U.S. Cl. CPC A61L 27/54; A61L 27/227; A61L 27/24; A61L 27/3675; A61L 27/3834; A61L 2300/252; A61L 2300/414; A61L 2300/426; A61L 2300/602; A61L 2300/622; A61L 2400/12; A61L 2430/32

(57) ABSTRACT

The present disclosure discloses a programmed controllable sustained-release bionic spinal cord graft, as well as a preparation method and application thereof, belonging to the field of medical biological materials. The preparation steps are as follows: synthesizing a micro-nano oriented fiber scaffolds with grooves based on recombinant NT3, silk fibroin, bovine collagen and nerve growth factor (NGF); jointly-constructing the programmed controllable sustained-release bionic spinal cord graft with the programmed sustained-release system of GelMA drug-loaded microspheres containing cyclosporin a in combination with the decellularized matrix derived from the human umbilical cord mesenchymal stem cells and the exosomes. The present disclosure integrates multiple technologies such as bionic tissue engineering, microfluidic technology, controllable sustained-release technology of drug-loaded microspheres, decellularized matrix, exosomes, which is conducive to the repair of spinal cord injury, possesses favorable biocompatibility, can promote the targeted growth of nerve axons, can exert immune regulation in the early stage after spinal cord injury, improves the microenvironment for nerve regeneration after spinal cord injury, and facilitates nerve regeneration and functional reconstruction after spinal cord injury.

### TECHNICAL FIELD

The present disclosure belongs to the technical field of medical biomaterials, and specifically relates to a programmed controllable sustained-release bionic spinal cord graft as well as a preparation method and application thereof.

### BACKGROUND

Spinal cord injury is a serious and disabling disease that commonly leads to irreversible damage to sensory and motor functions. Spinal cord injury can cause a series of complications such as urinary dysfunction, leg muscle weakness and atrophy, which seriously affect the patient's quality of life.

The dysregulation of neurotransmitters, activation of proinflammatory responses, cellular apoptosis, release of neurotoxic saturated lipids, impaired angiogenesis, inhibited axonal outgrowth, reconstruction of extracellular matrix structure, and fibrosis and scar formation after spinal cord injury are the primary causes of the difficulty in spinal cord injury repair. With the development of technologies such as bionic tissue materials, stem cells and exosomes, in addition, the technology of controlling physical parameters such as electricity, magnetism and ultrasound is constantly innovating and applied to the field of neural regeneration. These advanced new strategies and technologies have brought new treatment options for spinal cord injury repair. At present, the technology of treating spinal cord injury by utilizing stem cell therapy, biomaterial transplantation and electromagnetic stimulation has entered the clinical trial stage, but the clinical recovery effect is limited and clinical promotion is difficult. In view of the complex pathological mechanism following spinal cord injury, a single therapeutic approach is difficult to achieve ideal therapeutic effects after spinal cord injury.

### SUMMARY

### Technical Problems to Be Solved

In view of the deficiencies in the prior art, the present disclosure aims to solve the technical problems of limited clinical recovery effect, great difficulty in clinical popularization, and difficulty in achieving an ideal therapeutic effect after spinal cord injury by a single therapeutic approach, and provides a programmed controllable sustained-release bionic spinal cord graft as well as a preparation method and application thereof, which has the effects of spinal cord injury repair and functional reconstruction, and is conducive to nerve regeneration after spinal cord injury.

### Technical Solutions

To achieve the above objectives, the present disclosure is implemented through the following technical solutions.

Provided is a programmed controllable sustained-release bionic spinal cord graft. The programmed controllable sustained-release bionic spinal cord graft is prepared by adopting bionic tissue materials fabricated from factors beneficial to spinal cord injury repair, a silk fibroin and a bovine collagen, in combination with an immune drug, a decellularized matrix and exosomes to obtain the programmed controllable sustained-release bionic spinal cord graft; the factors beneficial to spinal cord injury repair are recombinant NT3 and neurotrophic factor β-NGF; and the bionic tissue material is micro-nano oriented fiber scaffolds with grooves.

Furthermore, steps for preparing the micro-nano oriented fiber scaffolds with grooves are as follows.

In Step 1, the silk fibroin and the bovine collagen are mixed in sterile water in a mass fraction ratio ranging from 100 to 500:1 to prepare a silk fibroin solution with a concentration ranging from 5 wt % to 20 wt %.

In Step 2, β-NGF ranging from 0.3 mL to 1.5 mL with an initial concentration of 15 μg/mL and recombinant NT3 ranging from 1 μL to 10 μL with an initial concentration of 0.6 ng/ml are added to a silk fibroin solution ranging from 0.7 mL to 2.5 mL, pipetted gently, mixed uniformly, and a silk fibroin blend solution is prepared.

In Step 3, the silk fibroin blend solution ranging from 100 μL to 1000 L are taken to wet and a gap of a PDMS membrane mold is filled, and then the silk fibroin blend solution ranging from 1 mL to 5 mL is added dropwise, poured into a mold with grooves, and flattened or leveled naturally.

In Step 4, the mold with the silk fibroin blend solution is flattened in a clean culture dish, naturally dried in a clean bench for a duration from 12 hours to 48 hours, and placed in an ethanol solution ranging from 70% to 100% for curing for a duration from 12 hours to 48 hours after demoulding. After the fiber is cured, dried naturally for a duration from 12 hours to 48 hours, and the micro-nano oriented fiber scaffolds with grooves are obtained.

In Step 5, before use, a PBS solution ranging from 200 μL to 1000 μL is taken, a β-NGF solution ranging from 1 μL to 500 μL with an initial concentration of 100 μg/mL and a recombinant NT3 ranging from 1 μL to 10 μL with an initial concentration of 0.6 ng/ml are added, pipetted gently, mixed uniformly, then the prepared micro-nano oriented fiber scaffolds with grooves are added and soaked overnight at 4 °C; then the fiber scaffolds are straightened in a manner of taking every 10 fiber scaffolds to 50 fiber scaffolds as one bundle for subsequent experiments.

Furthermore, in Step 2, a final concentration of recombinant NT3 ranges from 0.15 ng/ml to 6 ng/mL, and a final concentration of the β-NGF ranges from 7.5 μg/mL to 150 μg/mL.

Furthermore, a length of the groove in the mold with grooves ranges from 1 cm to 10 cm, and a cross-sectional shape of the groove is an inverted T-shape. The inverted T-shape is composed of a horizontal flange and a vertical web. A total horizontal length of the flange ranges from 50 μm to 500 μm, a thickness of the flange ranges from 20 μm to 50 μm, a height of the web ranges from 20 μm to 250 μm, and a width is permanently less than the total horizontal length of the flange.

Furthermore, the immune drug is GelMA microspheres CsA-GelMA containing cyclosporine a, a concentration of GelMA microspheres range from 5% to 20%, a concentration of cyclosporine a range from 10 mM to 200 mM. The decellularized matrix is a decellularized matrix derived from human umbilical cord mesenchymal stem cells, and the exosomes are exosomes derived from the human umbilical cord mesenchymal stem cells.

Furthermore, a method for preparing the GelMA microspheres CsA-GelMA containing cyclosporine a includes following steps.

In Step a, a GelMA solution is prepared.

In Step b, a uniform mixture of cyclosporine a and the GelMA solution is prepared. Under sterile conditions, CsA ranging from 12 mg to 120 mg is weighed and dissolved in the GelMA solution ranging from 5% to 20% prepared in Step a, and the solute is evenly dispersed by ultrasound at 37 °C, and a CsA-GelMA mixture with a concentration ranging from 10 mM to 100 mM is obtained.

In Step c, a single emulsion droplet microfluidic device is prepared.

In Step d, preparation of cyclosporine a and GelMA microspheres: a syringe containing the CsA-GelMA mixture and a syringe containing corn oil are placed on a syringe pump respectively, and connected to a microfluidic chip through a PE tube. A flow rate of the syringe containing the CsA-GelMA mixture ranges from 1 mL/h to 10 mL/h, and a flow rate of the syringe containing the corn oil ranges from 20 mL/h to 40 mL/h. A stable microsphere generation is observed under a microsphere, and then the microspheres are collected into a cell culture dish.

In Step e, the microspheres are collected and cured under a strong ultraviolet irradiation for a duration from 30 s to 300 s. The curing of the microspheres is checked under a microscope, and a diameter of the microspheres is measured within a range from 50 μm to 250 μm.

In Step f, the corn oil on a surface of the microspheres is eluted with ethanol ranging from 70% to 100% and the CsA-GelMA microspheres are collected.

Further provided is a method for preparing the programmed controllable sustained-release bionic spinal cord graft. The method includes the following steps. Under a condition of 4 °C, the CsA-GelMA microspheres are mixed with the micro-nano oriented fiber scaffolds with grooves. The CsA-GelMA microspheres are self-assembled on the micro-nano oriented fiber scaffolds with grooves, and a scaffold-microsphere morphology is formed. A decellularized matrix HUC-MSCs-ECM derived from the human umbilical cord mesenchymal stem cells is wrapped around the scaffold-microsphere with the scaffold-microsphere as an axis, and the scaffold-microsphere morphology is wrapped with the decellularized matrix HUC-MSCs-ECM derived from the human umbilical cord mesenchymal stem cells, the decellularized matrix derived from the human umbilical cord mesenchymal stem cells is wrapped for 1 layer to 10 layers with the micro-nano oriented scaffolds with grooves as the axis, an initial morphology of the programmed controllable sustained-release bionic spinal cord graft is prepared after freeze-drying. Exosomes derived from the human umbilical cord mesenchymal stem cells HUC-MSCs-EXO are collected by an ion exchange method. The initial morphology of the programmed controllable sustained-release bionic spinal cord graft is immersed in the extracted HUC-MSCs-EXO solution, the exosomes derived from the human umbilical cord mesenchymal stem cells HUC-MSCs-EXO are absorbed after freeze-drying. Time for absorbing the exosomes derived from the human umbilical cord mesenchymal stem cells ranges from 1 hour to 12 hours before use, and the programmed controllable sustained-release bionic spinal cord graft is finally obtained.

The present disclosure further discloses an application of any of the above-mentioned programmed controllable sustained-release bionic spinal cord grafts or the programmed controllable sustained-release bionic spinal cord grafts prepared by the above-mentioned preparation method in materials for repairing spinal cord injuries.

Furthermore, the micro-nano oriented fiber scaffolds with grooves are biodegradable scaffolds. The micro-nano oriented fiber scaffolds with grooves are integrated with the recombinant NT3, the silk fibroin, the neurotrophic factor β-NGF and the bovine collagen, have a good biocompatibility, and the factors conducive to spinal cord injury repair achieve a programmed controllable sustained-release along with a degradation of the scaffold material in vivo, thereby providing a suitable microenvironment for a nerve regeneration, providing a guidance and a nerve protection for a growth of nerve axons, and promoting the nerve regeneration. The GelMA microspheres containing cyclosporine a are capable of a programmed sustained-release of the cyclosporin a, and exert an immunomodulatory effect in an early stage of the spinal cord injury. The scaffold-microsphere morphology is wrapped with the decellularized matrix HUC-MSCs-ECM derived from the human umbilical cord mesenchymal stem cells, providing a beneficial microenvironment for the growth of nerve cells, as well as having effects of guiding and oriented growth.

Principle Explanation: In the present disclosure, the micro-nano oriented fiber scaffolds with grooves are formed by adopting recombinant NT3, silk fibroin fused with bovine collagen and neurotrophic factor NGF, based on biomaterials, seed cells, active factors, cell matrix and regenerative microenvironment; a programmed controllable sustained-release bionic spinal cord graft is designed and constructed, in combination with microfluidic technology for the programmed sustained-release of the immune drug cyclosporin a, in further conjunction with the decellularized matrix and exosome technology derived from human umbilical cord mesenchymal stem cells. In the present disclosure, a mouse spinal cord semi-defect experiment is carried out, demonstrating that the programmed controllable sustained-release bionic spinal cord graft provided by the present disclosure has the effect of repairing mouse spinal cord injury and functional reconstruction, which provides new treatment plans and strategies for clinical research and treatment, and brings innovative new medical technologies to the repair, regeneration and functional reconstruction of human spinal cord injuries.

### Beneficial Effects

The present disclosure provides a programmed controllable sustained-release bionic spinal cord graft, a preparation method and application, which have the following beneficial effects in comparison with the prior art.
1. In the present disclosure, silk fibroin fused with bovine collagen is adopted to construct a micro-nano oriented fiber scaffold with grooves, which possesses favorable biocompatibility and biodegradability, and exerts a guiding effect on axon growth.
2. The micro-nano oriented fiber scaffold with grooves of the present disclosure contains recombinant NT3 and β-NGF, which can be sustainedly released for a long time with the degradation of the scaffold material, thereby exerting neuroprotective effects and promoting nerve regeneration.
3. The GelMA microspheres encapsulating cyclosporine a adopted in the present disclosure implement programmed sustained-release of cyclosporine a, possess good biocompatibility and biodegradability, and play a role in regulating immune response in the early stage of spinal cord injury.
4. The decellularized matrix derived from human umbilical cord mesenchymal stem cells adopted in the present disclosure retains various important components and main frameworks of the extracellular matrix, which is conducive to cell adhesion and axon regeneration.
5. The exosomes derived from human umbilical cord mesenchymal stem cells adopted in the present disclosure exert the effects of immunomodulation and promoting nerve regeneration in spinal cord injury.
6. The decellularized matrix and exosomes derived from human umbilical cord mesenchymal stem cells adopted in the present disclosure facilitate the clinical transformation of programmed controllable sustained-release bionic spinal cord grafts.
7. In the present disclosure, multiple technologies are combined to treat spinal cord injury from multiple aspects, which achieves spinal cord injury repair and functional reconstruction in a mouse spinal cord hemi-defect model.

### BRIEF DESCRIPTION OF THE DRAWINGS

FIG. 1 illustrates a morphological diagram of a freeze-dried programmed controllable sustained-release bionic spinal cord graft provided by the present disclosure.

FIGS. 2A and 2B illustrates a diagram showing that the programmed controllable sustained-release bionic spinal cord graft provided by the present disclosure has good biocompatibility, FIG. 2A illustrates a morphological diagram of the programmed controllable sustained-release bionic spinal cord graft provided by the present disclosure in a mouse with a 3 mm semi-defect of the spinal cord at 3 W, 6 W, 9 W, and 12 W; FIG. 2B illustrates a cytotoxicity experiment (CCK8) of PC12 cells cultured for 24 h in an extract solution of the programmed controllable sustained-release bionic spinal cord graft of the present disclosure.

FIGS. 3A and 3B illustrates a function analysis diagram of motions of an adult B6 mouse with a 3 mm semi-defect of the spinal cord repaired by the programmed controllable sustained-release bionic spinal cord graft provided by the present disclosure, FIG. 3A illustrates a behavioral photograph of the experimental mouse; and FIG. 3B illustrates a gait imprint of the experimental mouse catwalk hind limbs.

FIGS. 4A-4D illustrates a functional analysis diagram of hind limbs of the adult B6 mice with the 3 mm semi-defect of the spinal cord repaired by the programmed controllable sustained-release bionic spinal cord graft provided by the present disclosure, FIG. 4A illustrates a morphological diagram of hind limb muscles of the experimental mice; FIG. 4B illustrates a statistics of a muscle wet weight ratio ***p<0.001, ****p<0.001, Student's t-test analysis; FIG. 4C illustrates a laser speckle blood flow imaging; FIG. 4D illustrates statistics of a mechanical pain threshold of right hind limbs of the experimental mice *p<0.05, **p<0.01, Student's t-test analysis.

FIGS. 5A-5C illustrates a functional analysis diagram of a bladder of the adult B6 mouse with the 3 mm semi-defect of the spinal cord repaired by the programmed controllable sustained-release bionic spinal cord graft provided by the present disclosure, FIG. 5A illustrates a bladder morphology of the experimental mouse at 12 W; FIG. 5B illustrates a bladder volume statistics of the experimental mouse at 12 W * ****p<0.001, Student's t-test analysis; FIG. 5C illustrates a HE staining of the bladder from the experimental mouse at 12 W, with a scale bar of 200 μm.

FIG. 6 illustrates an analysis diagram of a spinal cord immunity and a glial scar of the adult B6 mouse with the 3 mm semi-defect of the spinal cord repaired by the programmed controllable sustained-release bionic spinal cord graft provided by the present disclosure. Tissue immunofluorescence staining is performed at 3 W, 6 W, 9 W, and 12 W after surgery, respectively. Green represents IBA-1 labeled microglia, red represents GFAP labeled astrocytes, and blue represents DAPI labeled cell nuclei. The staining scale is 200 μm.

FIG. 7 illustrates an analysis diagram of a spinal cord axon and a vascular regeneration of the adult B6 mouse with the 3 mm semi-defect of the spinal cord repaired by the programmed controllable sustained-release bionic spinal cord graft provided by the present disclosure. Tissue immunofluorescence staining is performed at 3 W, 6 W, 9 W, and 12 W after surgery, respectively. Red represents TUJ1-labeled axons, green represents CD31-labeled blood vessels, and blue represents DAPI-labeled cell nuclei. The staining scale is 200 μm.

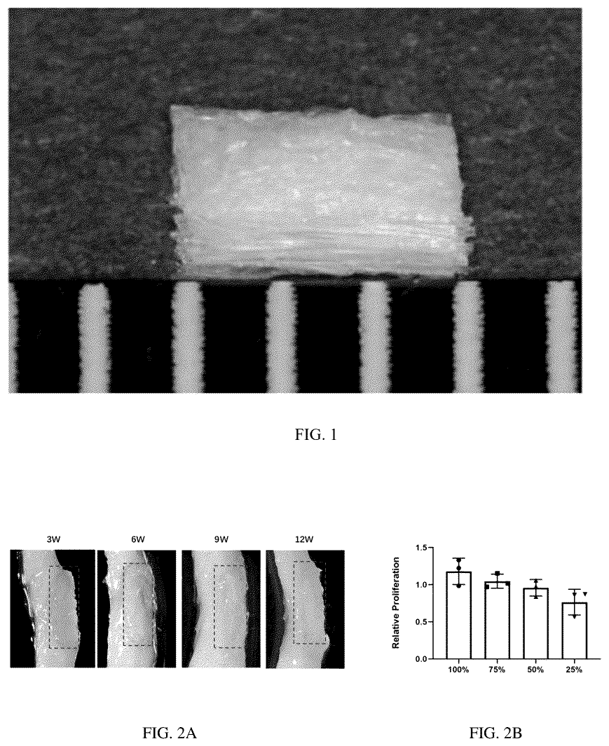

### DETAILED DESCRIPTION OF THE EMBODIMENTS

The present disclosure is described in detail below in conjunction with specific examples. The following examples will help those skilled in the art to further understand the present disclosure, but shall not intended to limit the present disclosure in an arbitrary form. It should be noted that, for those of ordinary skill in the art, a plurality of variations and improvements may be made without departing from the concept of the present disclosure. These all belong to the protection scope of the present disclosure.

### Example 1

The programmed controllable sustained-release bionic spinal cord graft is prepared by adopting bionic tissue materials fabricated from factors beneficial to spinal cord injury repair, in combination with an immune drug to obtain the programmed controllable sustained-release bionic spinal cord graft. The factors beneficial to spinal cord injury repair include recombinant NT3, silk fibroin, neurotrophic factor β-NGF and bovine collagen. The bionic tissue material is a micro-nano oriented scaffold with grooves. And the preparation steps of the micro-nano oriented scaffold with grooves are as follows.

In Step 1, bovine collagen is cut into powder, water is added. The bottle is weighed with contents, and then placed in a sterilization pot at 105 °C for 10 min. After taken out and cooled to room temperature, the surface moisture is wiped off and the bottle is weighed again to replenish the water lost during sterilization. Silk fibroin is weighed, sterile deionized water is added, a rotor is added to stir for 30 minutes, then the sterilized bovine collagen is added. The mass fraction ratio of silk fibroin to bovine collagen is controlled within a range from 100 to 500:1, and the mixture is continuously stirred for 30 min. The mixed solution is allowed to stand in a refrigerator at 4 °C for defoaming, and a silk fibroin solution with a concentration ranging from 5 wt % to 20 wt % is obtained.

In Step 2, β-NGF ranging from 0.3 mL to 1.5 mL with an initial concentration of 100 μg/mL and recombinant NT3 ranging from 1 μL to 10 μL with an initial concentration of 0.6 ng/ml are added into silk fibroin solution ranging from 0.7 mL to 2.5 mL, pipetted gently, mixed uniformly, to prepare a silk fibroin blend solution. The final concentration of the recombinant NT3 is 0.6 ng/ml, and the final concentration of-NGF is 15 μg/mL.

In Step 3, the PDMS membrane mold and cell scraper are sterilized and dried. 400 μL of the silk fibroin blend solution is taken to wet and fully fill the gaps of the PDMS membrane mold. Subsequently, 1.5 mL of the silk fibroin blend solution is dropwise added and poured onto the mold with grooves, flattened or leveled naturally. A length of grooves in the mold with grooves ranges from 1 cm to 10 cm. The cross-sectional shape of each groove is an inverted T-shape, which is composed of a horizontal flange and a vertical web. A total horizontal length of the flange ranges from 50 μm to 500 μm, a thickness of the flange ranges from 20 μm to 50 μm; a height of the web ranges from 20 μm to 250 μm, and the width of the web is permanently less than the total horizontal length of the flange.

In Step 4, the mold with the silk fibroin blend solution is flattened in a clean culture dish, naturally dried in a clean bench for 24 hours. After demolding, the mold is placed in an ethanol solution ranging from 70% to 100% for 24 h for curing. After fiber curing, the absolute ethanol is extracted, and the material is naturally dried for 24 h in an unventilated environment. Upon complete drying, the fibers are peeled off from the mold using microscopic straight forceps in a super-clean bench and placed into a clean culture dish, followed by storage in a refrigerator at −20 °C for later use. Consequently, the micro-nano oriented fiber scaffold with grooves is obtained.

In Step 5, before use, in a clean bench, the PBS solution ranging from 200 μL to 1000 μL is take, the β-NGF solution ranging from 1 μL to 500 μL with an initial concentration of 100 μg/mL and the recombinant NT3 ranging from 1 μL to 10 μL with an initial concentration of 0.6 ng/ml are added, pipetted gently, mixed uniformly, then the prepared micro-nano oriented fiber scaffold with grooves is added and soaked overnight at 4 °C. Then the fiber scaffolds are straightened in a manner of taking every 10 fiber scaffolds to 50 fiber scaffolds as one bundle for subsequent experiments.

### Example 2

The method of preparing GelMA microspheres CsA-GelMA containing cyclosporine a includes the following steps.

In Step a, GelMA solution is prepared. Under sterile conditions, GelMA solid powder ranging from 0.05 g to 2 g is weighed and dissolved in 1 mL of sterile 1× PBS. The mixture is heated at 50 °C to assist dissolution and subjected to high-speed centrifugation for defoaming to prepare a clear and bubble-free GelMA solution ranging from 5% to 20%, which is maintained at 37 °C to prevent condensation. To endow the solution with photocurable properties, lithium phenyl (2,4,6-trimethylbenzoyl) phosphinate (LAP) is added into the GelMA solution at a ratio of 1:10000 and mixed uniformly. The resulting solution is filtered for bacteria filtration through a 0.22 μm filter head and stored away from light for subsequent use.

In Step b, a uniform mixture of CsA cyclosporine a and GelMA solution is prepared: Under sterile conditions, CsA ranging from 12 mg to 120 mg is weighed and dissolved in the GelMA solution ranging from 5% to 20% prepared in Step a, and the solute is evenly dispersed by ultrasound at 37 °C to finally obtain a CsA-GelMA mixture with a concentration ranging from 10 mM to 100 mM.

In Step c, a single emulsion droplet microfluidic device is prepared. Medicinal-grade corn oil is filtered through a 0.22 μm filter head for bacteria filtration for subsequent use. A chip is assembled in a clean bench, an inner diameter of the inner phase tube is 120 μm, an inner diameter of the outer phase tube is 580 μm, and an inner diameter of the square tube is 1.05 mm. The tubes are connected to construct a microfluidic device.

In Step d, cyclosporine a and GelMA microspheres are prepared: CsA-GelMA mixed solution and corn oil are respectively loaded into a sterile 1 ml syringe and a sterile 5 mL syringe. And the syringes loaded with CsA-GelMA mixed solution and the syringes loaded with corn oil are respectively placed on syringe pumps, and are connected to the microfluidic chip through PE tubes. A flow rate of the syringe loaded with CsA-GelMA mixed solution ranges from 1 mL/h to 10 mL/h, and a flow rate of the syringe loaded with corn oil ranges from 20 mL/h to 40 mL/h. A stable microsphere generation is observed under a microsphere, and then the microspheres are collected into a cell culture dish.

In Step e, the microspheres are collected and cured under strong ultraviolet irradiation for 180 seconds. The curing of the microspheres is checked under a microscope, and a diameter of the microspheres is measured within a range from 100 μm to 250 μm.

In Step f, the corn oil on the surface of the microspheres is eluted with anhydrous ethanol and the CsA-GelMA microspheres are collected.

### Example 3

The self-assembly of CsA-GelMA microspheres and the micro-nano oriented fiber scaffold with grooves includes the following steps.

The self-assembly of microspheres and scaffolds is completed in a clean bench under a condition of 4 °C. Every 10 fibers to 50 fibers are bundled into one bundle of fiber scaffolds, and CsA-GelMA microspheres are added. The microspheres are evenly distributed inside and on the surface of the scaffolds by gently mixing.

### Example 4

The encapsulation of HUC-MSCs-ECM is carried out according to the following steps.

The decellularized matrix HUC-MSCs-ECM derived from human umbilical cord mesenchymal stem cells is wrapped around the scaffold-microspheres with the scaffold-microspheres as the axis. The scaffold-microsphere structure is wrapped with decellularized matrix HUC-MSCs-ECM derived from human umbilical cord mesenchymal stem cells. The decellularized matrix derived from human umbilical cord mesenchymal stem cells is wrapped for 1 layer to 10 layers with the micro-nano oriented scaffold with grooves as the axis, which provides the necessary pathway for the growth of nerve cells, as well as has effects of the guiding and oriented growth.

### Example 5

The steps of constructing the final programmed controllable sustained-release bionic spinal cord graft are as follows.

The initial morphology of a programmed controllable sustained-release bionic spinal cord graft is prepared after freeze-drying. As illustrated in FIG. 1, the figure illustrates a morphological diagram of the initial morphology of a programmed controllable sustained-release bionic spinal cord graft taken under an optical microscope.

Exosomes derived from human umbilical cord mesenchymal stem cells HUC-MSCs-EXO are collected by an ion exchange method. After freeze-drying, the initial morphology of programmed controllable sustained-release bionic spinal cord graft is immersed in the extracted HUC-MSCs-EXO solution, and exosomes derived from human umbilical cord mesenchymal stem cells HUC-MSCs-EXO are absorbed after freeze-drying. The time for absorbing exosomes derived from human umbilical cord mesenchymal stem cells ranges from 1 hour to 12 hours before use, and a programmed controllable sustained-release bionic spinal cord graft is obtained.

### Example 6

A 3 mm semi-defect of the spinal cord in mice is repaired by programmed controllable sustained-release bionic spinal cord grafts.

The 3 mm semi-defect of the spinal cord in adult B6 mice is repaired by a programmed controllable sustained-release bionic spinal cord graft. The recovery of motor function after spinal cord injury repair is evaluated via BMS behavioral score, CatWalk, muscle wet weight ratio measurement, lower limb blood flow Doppler imaging and mechanical pain assessment.

A 3 mm semi-defect model of the right spinal cord in adult B6 mice is established and the mice are randomly divided into two groups. In the bionic spinal cord graft group (material group), the spinal cord semi-defects of B6 mice are repaired with the programmed controllable sustained-release bionic spinal cord graft. In the control group (NC group), no intervention is performed after the spinal cord hemisection injury in B6 mice.

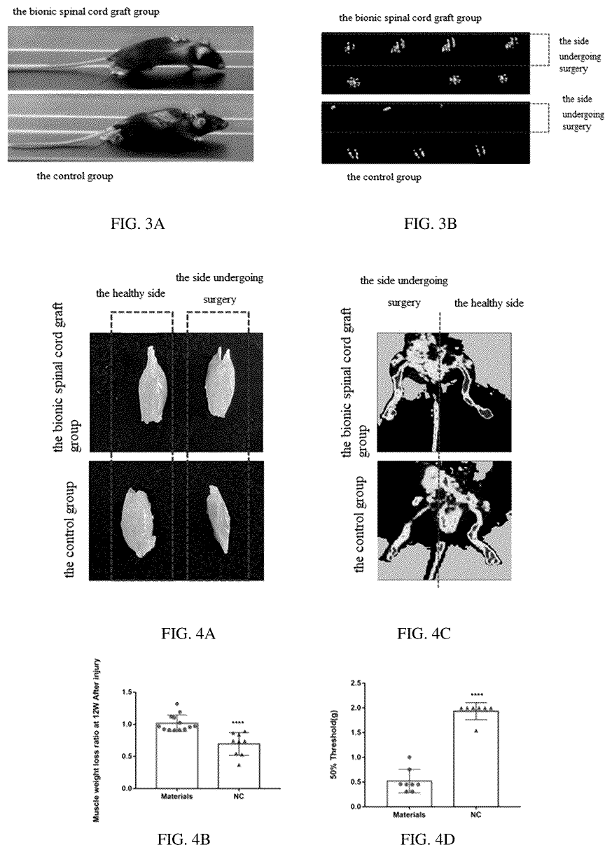

BMS behavioral assessment and scoring are performed at 3 weeks, 6 weeks, 9 weeks, and 12 weeks after surgery. As illustrated in FIGS. 3A and 3B, FIG. 3A illustrates an image of the BMS scoring at 12 weeks after surgery provided by an embodiment of the present disclosure. In the programmed controllable sustained-release bionic spinal cord graft group, the mice frequently walk on the right hind limbs, and the trunk remains stable during grounding and lifting. In the control group, the hind limbs of mice are dragged, and independent walking cannot be realized.

FIG. 3B illustrates the result of the calkwalk experiment. Clear footprints of the right hind limbs are observed in the programmed controllable sustained-release bionic spinal cord graft group, with uniform plantar pressure distribution. In the control group, the right hind limbs of mice are dragged, plantar walking cannot be realized, and almost no valid footprint records are obtained.

The detection on the wet weight ratio of the target muscle after surgery is an important indicator for evaluating the reconstruction of motor function. As illustrated in FIG. 4A and FIG. 4B, it is a schematic diagram of the wet weight ratio of the target muscle provided in an embodiment of the present disclosure. As illustrated in FIG. 4A, it is an appearance morphology diagram of the two groups of target muscles and the corresponding muscles on the healthy side. As illustrated in FIG. 4B, it is an analysis of the wet weight ratio of the gastrocnemius muscles of the two groups, ***p<0.001, ****p<0.001. It can be seen from the figure that at 12 weeks after surgery, the wet weight ratios of the gastrocnemius muscles of the programmed controllable sustained-release bionic spinal cord graft group and the control group are 1.0±0.25 and 0.6±0.25, respectively, with a statistically significant difference between the two groups.

Spinal cord injury can lead to unstable blood flow and weakened blood supply in the hind limbs of mice, which affects the recovery of motor function. Laser speckle blood flow imaging of the blood flow of the hind limb on the operated side is an indicator for evaluating the recovery of motor function. As illustrated in FIG. 4C, the laser speckle blood flow imaging diagram provided by an embodiment of the present disclosure illustrates that sufficient blood perfusion of the operative right hind limb is exhibited in the programmed controllable sustained-release bionic spinal cord graft group, while blood supply is obviously insufficient in the control group.

Postoperative mechanical pain detection is an important indicator for evaluating the recovery of sensory function. As illustrated in FIG. 4D, a mechanical pain statistical graph provided in an embodiment of the present disclosure illustrates that the pain thresholds of the programmed controllable sustained-release bionic spinal cord graft group and the control group are 0.5±0.25 and 1.9±0.25, respectively, with a statistically significant difference between the two groups (***p<0.001, ****p<0.001) .

### Example 7

The programmed controllable sustained-release bionic spinal cord grafts is adopted to promote bladder function recovery after 3 mm hemi-defect of the spinal cord in adult B6 mice.

The programmed controllable sustained-release bionic spinal cord graft is adopted to promote the recovery of bladder function after 3 mm hemi-defect of the spinal cord in adult B6 mice. The recovery of bladder function is evaluated by bladder volume and bladder HE staining.

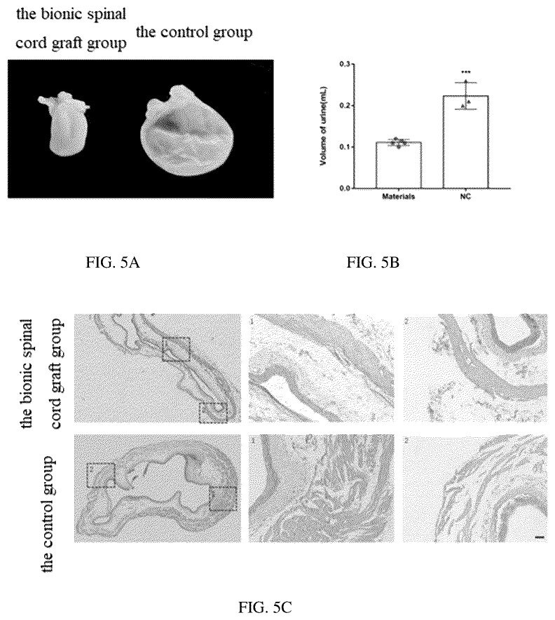

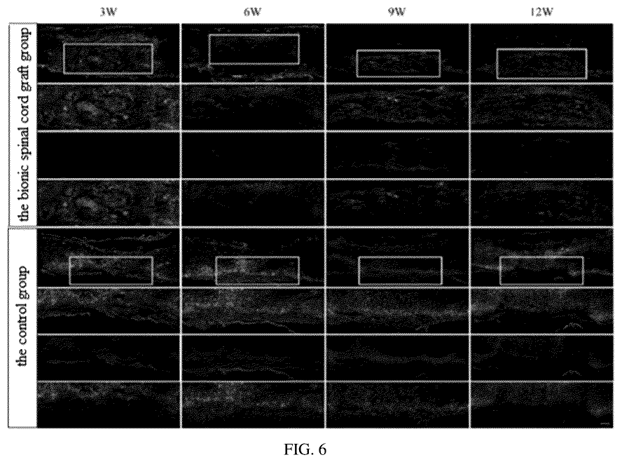

FIG. 5A illustrates a morphological image of the bladder of mice in the programmed controllable sustained-release bionic spinal cord graft group and the control group in 12 weeks after surgery.

FIG. 5B illustrates a statistical diagram of the bladder volume of the programmed controllable sustained-release bionic spinal cord graft group and the control group in 12 weeks after surgery. The statistical diagram illustrates that the bladder volumes of the bionic spinal cord graft group and the control group are 0.1±0.01 and 0.2±0.2, respectively, with statistical differences (***p<0.001, ****p<0.001) . The specific steps are as follows.

The mice are anesthetized with mouse anesthetic (tribromoethanol). The bladder is exposed, and normal saline is slowly injected into the bladder of mice with completely drained urine by using a syringe. The volume of normal saline injected when the bladder is fully distended to the maximum capacity is recorded.

FIG. 5C illustrates the HE staining images of the bladder in 12 weeks after surgery provided in this example.

Result analysis indicates that the bladder wall of the control group is thickened and the muscle bundles are disordered, while these abnormalities are significantly improved in the programmed controllable sustained-release bionic spinal cord graft group.

### Example 8

The programmed controllable sustained-release biomimetic spinal cord grafts is adopted to reduce inflammatory responses and glial scarring after 3 mm hemi-defects of the spinal cord in adult B6 mice.

The programmed controllable sustained-release bionic spinal cord graft is adopted to reduce the inflammatory response and glial scar after 3 mm hemi-defect of the spinal cord in adult B6 mice. As illustrated in FIG. 6, the tissue immunofluorescence images of microglia (green), astrocytes (red) and cell nuclei (blue) is provided in this example.

### Example 9

The programmed controllable slow-release bionic spinal cord grafts is adopted to promote axonal and vascular regeneration after 3 mm hemi-defect of the spinal cord in adult B6 mice.

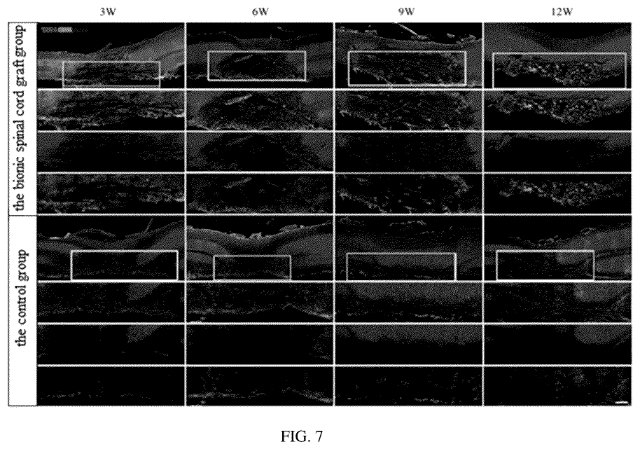

The programmed controllable slow-release bionic spinal cord graft is adopted to promote axon and blood vessel regeneration after 3 mm hemi-defect of the spinal cord in adult B6 mice. As illustrated in FIG. 7, the tissue immunofluorescence staining images of axons (red), blood vessels (green) and cell nuclei (blue) are provided in this example.

In the present disclosure, the programmed controllable sustained-release bionic spinal cord graft used does not contain exogenous toxic substances introduced due to the preparation process, exhibiting excellent biocompatibility and biodegradability; the biodegradable scaffold, microfluidic technology, extracellular matrix and exosome technology are used and a tubular structure is constructed, providing necessary directional growth for nerve cell growth; the used nerve growth factor, Cyclosporin A (CsA) microspheres, decellularized matrix and exosomes can regulate the microenvironment after spinal cord injury, effectively promoting nerve regeneration and functional recovery.

The examples are provided merely for the convenience of understanding the present disclosure, rather than limiting the technical method thereof. Those skilled in the art can readily modify the technical process or apply the technical solution to other embodiments without creative efforts. All modifications and applications falling within the same technical scope and claims of the present disclosure shall be deemed to be included within the protection scope of the present disclosure.

1. A programmed controllable sustained-release bionic spinal cord graft, wherein the programmed controllable sustained-release bionic spinal cord graft is prepared by adopting bionic tissue materials fabricated from factors beneficial to spinal cord injury repair, a silk fibroin, and a bovine collagen, in combination with an immune drug, a decellularized matrix, and exosomes to obtain the programmed controllable sustained-release bionic spinal cord graft; the factors beneficial to spinal cord injury repair are recombinant NT3 and neurotrophic factor β-NGF;
the bionic tissue material is micro-nano oriented fiber scaffolds with grooves.

2. The programmed controllable sustained-release bionic spinal cord graft according to claim 1, wherein steps for preparing the micro-nano oriented fiber scaffolds with grooves are:
Step 1: mixing the silk fibroin and the bovine collagen in sterile water in a mass fraction ratio ranging from 100 to 500:1 to prepare a silk fibroin solution with a concentration ranging from 5 wt % to 20 wt %;
Step 2: adding β-NGF ranging from 0.3 mL to 1.5 mL with an initial concentration of 15 μg/mL and recombinant NT3 ranging from 1 μL to 10 μL with an initial concentration of 0.6 ng/ml to a silk fibroin solution ranging from 0.7 mL to 2.5 mL, pipetting gently, mix uniformly, and preparing a silk fibroin blend solution;
Step 3: taking the silk fibroin blend solution ranging from 100 μL to 1000 μL to wet and filling a gap of a PDMS membrane mold, then adding the silk fibroin blend solution ranging from 1 mL to 5 mL dropwise, pouring into a mold with grooves, flattening or leveling naturally;
Step 4: flattening the mold with the silk fibroin blend solution in a clean culture dish, naturally drying in a clean bench for a duration from 12 hours to 48 hours, and placing in an ethanol solution ranging from 70% to 100% for curing for a duration from 12 hours to 48 hours after demoulding, during naturally for a duration from 12 hours to 48 hours after curing the fiber, and obtaining the micro-nano oriented fiber scaffolds with grooves; and
Step 5: before use, taking a PBS solution ranging from 200 μL to 1000 μL, adding a β-NGF solution ranging from 1 μL to 500 μL with an initial concentration of 100 μg/mL and a recombinant NT3 ranging from 1 μL to 10 μL with an initial concentration of 0.6 ng/mL, pipetting gently, mix uniformly, then adding the prepared micro-nano oriented fiber scaffolds with grooves, and soaking overnight at 4 °C; then straightening the fiber scaffolds in a manner of taking every 10 fiber scaffolds to 50 fiber scaffolds as one bundle for subsequent experiments.

3. The programmed controllable sustained-release bionic spinal cord graft according to claim 2, wherein in Step 2, a final concentration of the recombinant NT3 ranges from 0.15 ng/ml to 6 ng/ml, and a final concentration of the β-NGF ranges from 7.5 μg/mL to 150 μg/mL.

4. The programmed controllable sustained-release bionic spinal cord graft according to claim 2, wherein a length of the groove in the mold with grooves ranges from 1 cm to 10 cm, a cross-sectional shape of the groove is an inverted T-shape, and the inverted T-shape is composed of a horizontal flange and a vertical web; a total horizontal length of the flange ranges from 50 μm to 500 μm, a thickness of the flange ranges from 20 μm to 50 μm, a height of the web ranges from 20 μm to 250 μm, and a width is permanently less than the total horizontal length of the flange.

5. The programmed controllable sustained-release bionic spinal cord graft according to claim 1, wherein the immune drug is GelMA microspheres CsA-GelMA containing cyclosporine a, wherein a concentration of the GelMA microspheres ranges from 5% to 20%, a concentration of cyclosporine a ranges from 10 mM to 200 mM, the decellularized matrix is a decellularized matrix derived from human umbilical cord mesenchymal stem cells, and the exosomes are exosomes derived from the human umbilical cord mesenchymal stem cells.

6. The programmed controllable sustained-release bionic spinal cord graft according to claim 5, wherein a method for preparing GelMA microspheres CsA-GelMA containing cyclosporine a comprises following steps:
Step a: preparing a GelMA solution;
Step b: preparing a uniform mixture of cyclosporine a and the GelMA solution: under sterile conditions, weighing CsA ranging from 12 mg to 120 mg and dissolving in the GelMA solution ranging from 5% to 20% prepared in Step a, and evenly dispersing the solute by ultrasound at 37 °C, finally obtaining a CsA-GelMA mixture with a concentration ranging from 10 mM to 100 mM;
Step c: preparing a single emulsion droplet microfluidic device;
Step d, preparing cyclosporine a and GelMA microspheres: placing a syringe containing the CsA-GelMA mixture and a syringe containing corn oil on a syringe pump, respectively, and connecting to a microfluidic chip through a PE tube, observing a stable microsphere generation under a microscope before collecting the microspheres into a cell culture dish, wherein a flow rate of the syringe containing the CsA-GelMA mixture ranges from 1 mL/h to 10 mL/h, and a flow rate of the syringe containing the corn oil ranges from 20 mL/h to 40 mL/h ; ;
Step e: collecting the microspheres and curing under a strong ultraviolet irradiation for a duration from 30 s to 300 s, checking the curing of the microspheres under a microscope, and measuring a diameter of the microspheres within a range from 50 μm to 250 μm; and
Step f: eluting the corn oil on a surface of the microspheres with ethanol ranging from 70% to 100% and collecting the CsA-GelMA microspheres.

7. A method for preparing the programmed controllable sustained-release bionic spinal cord graft according to claim 1, wherein the method includes: under a condition of 4 °C, mixing the CsA-GelMA microspheres and the micro-nano oriented fiber scaffolds with grooves, self-assembling the CsA-GelMA microspheres on the micro-nano oriented fiber scaffolds with grooves, forming a scaffold-microsphere morphology, wrapping a decellularized matrix HUC-MSCs-ECM derived from the human umbilical cord mesenchymal stem cells around the scaffold-microsphere with the scaffold-microsphere as an axis, and wrapping the scaffold-microsphere morphology with the decellularized matrix HUC-MSCs-ECM derived from the human umbilical cord mesenchymal stem cells, preparing an initial morphology of the programmed controllable sustained-release bionic spinal cord graft after freeze-drying, collecting exosomes derived from the human umbilical cord mesenchymal stem cells HUC-MSCs-EXO by an ion exchange method; immersing the initial morphology of the programmed controllable sustained-release bionic spinal cord graft in the extracted HUC-MSCs-EXO solution, adsorbing the exosomes derived from the human umbilical cord mesenchymal stem cells HUC-MSCs-EXO after freeze-drying, finally obtaining the programmed controllable sustained-release bionic spinal cord graft, wherein the decellularized matrix derived from the human umbilical cord mesenchymal stem cells is wrapped for 1 layer to 10 layers with the micro-nano oriented scaffolds with grooves as the axis, and time for absorbing the exosomes derived from the human umbilical cord mesenchymal stem cells ranges from 1 hour to 12 hours before use.

8. An application of the programmed controllable sustained-release bionic spinal cord graft according to claim 1 or the programmed controllable sustained-release bionic spinal cord graft prepared by the preparation method in materials for repairing spinal cord injuries.

9. The application according to claim 8, wherein the micro-nano oriented fiber scaffolds with grooves are biodegradable scaffolds, and the micro-nano oriented fiber scaffolds with grooves are integrated with the recombinant NT3, the silk fibroin, the neurotrophic factor β-NGF and the bovine collagen, have a good biocompatibility, and the factors conducive to spinal cord injury repair achieve a programmed controllable sustained-release along with a degradation of the scaffold material in vivo, thereby providing a suitable microenvironment for a nerve regeneration, providing a guidance and a nerve protection for a growth of nerve axons, and promoting the nerve regeneration; the GelMA microspheres containing cyclosporine a are capable of a programmed sustained-release of the cyclosporin a, and exert an immunomodulatory effect in an early stage of the spinal cord injury; the scaffold-microsphere morphology is wrapped with the decellularized matrix HUC-MSCs-ECM derived from the human umbilical cord mesenchymal stem cells, providing a beneficial microenvironment for the growth of nerve cells, as well as having effects of guiding and oriented growth.
